(19) United States
(12) Patent Application Publication
(10) Pub. No.: US 2013/0317611 A1
(43) Pub. Date: Nov. 28, 2013

(54) USE OF A MEDICAL IMPLANT AS ADHESION BARRIER

(71) Applicant: Gelita AG, Eberbach (DE)

(72) Inventor: Steffen OESSER, Glucksburg (DE)

(73) Assignee: Gelita AG, Eberbach (DE)

(21) Appl. No.: 13/955,752

(22) Filed: Jul. 31, 2013

Related U.S. Application Data
(63) Continuation of application No. PCT/EP2012/052702, filed on Feb. 16, 2012.

(30) Foreign Application Priority Data
Feb. 16, 2011 (DE) 10 2011 004 239.3

(51) Int. Cl. A61F 2/02

(52) U.S. Cl. CPC A61F 2/02
USPC 623/11.11

(57) ABSTRACT

The present invention relates to use of a medical implant as an adhesion barrier. The medical implant is a sheet material which comprises at least one layer of a non-woven fabric of fibres of a crosslinked gelatin material.

### CROSS-REFERENCE TO RELATED APPLICATIONS

This patent application is a continuation of International Application Number PCT/EP2012/052702, filed Feb. 16, 2012, claiming the benefit of German Patent Application No. 10 2011 004 239.3, filed Feb. 16, 2011, which are each incorporated by reference.

The present invention relates to the use of a medical implant as an adhesion barrier.

Adhesion barriers are inserted into a patient's body after surgery to keep tissue layers separate and remain there with this function during the critical phase of wound healing. The adhesion barriers are preferably produced from materials which are degradable under physiological conditions, such that they may then be broken down by the metabolism over the course of time and excreted by the body, so obviating the need for surgical intervention to remove the adhesion barrier.

Adhesion barriers are primarily used in abdominal surgery, in particular for operations on the small or large intestine or rectum, for the complete and partial removal of the small or large intestine, in the removal of adhesions (adhesiolysis) and in general in open operations in the abdominal region where subsequent operations are anticipated. Further fields of application are gynaecology and urology, in particular in a myomectomy, in operations on the Fallopian tubes, in ovarian cyst removal and similar indications.

Using an adhesion barrier avoids unwanted adhesion of tissue layers after surgical intervention which would otherwise require further surgery to detach. In this way, many and varied complications following surgery are avoided and the need for surgical measures is minimised. Adhesions in this connection are taken to mean unwanted coalescences or pathological scarring as a consequence of surgical intervention.

Conventional adhesion barriers which form a physical barrier between affected tissue layers during the wound healing phase and are physiologically absorbed over the course of time substantially consist of a mixture of chemically modified anionic polysaccharides, namely hyaluronic acid derivatives and carboxymethylcellulose.

While the carboxymethylcellulose part of conventional adhesion barriers can be produced relatively inexpensively and with relatively high batch security, the properties of the second constituent which is functionally indispensable in conventional adhesion barriers, namely the hyaluronic acid derivative, are problematic. This substance is not only costly, but is also difficult to obtain in pure form, such that the properties of the adhesion barriers produced therewith, in particular the absorption times thereof, are variable and thus do not exhibit sufficient batch security.

The object of the present invention is to propose the use of a medical implant as an adhesion barrier which, in a similar manner to conventional biologically absorbable adhesion barriers, can remain in the patient's body and be physiologically degraded, while on the other hand being less costly to produce and exhibiting greater batch security.

Said object is achieved according to the invention in that the medical implant is a sheet material which comprises at least one layer of a non-woven fabric of fibres of a crosslinked gelatin material.

Thanks to the use of a crosslinked gelatin material, it is possible to dispense with hyaluronic acid and likewise also with carboxymethylcellulose, whereby on the one hand the hyaluronic acid derivative is replaced by a less costly material which can be produced with greater batch security and, on the other hand, a series of additional advantageous properties of the adhesion barrier are obtained.

The biocompatibility of gelatin-based materials is well known since gelatin, unlike collagen for instance, is a material with a largely defined composition which can be produced in the purity required for medical applications.

Furthermore, the absorption time of the adhesion barrier under physiological conditions can be specified across a broad window by the degree of crosslinking of the gelatin material, such that adhesion barriers with barrier function times of different lengths can be produced.

In addition to the general advantages which gelatin offers as a starting material for a medical implant, it has surprisingly been found that a non-woven fabric of fibres of a crosslinked gelatin material is particularly well suited to being an adhesion barrier. The efficacy of the medical implant used according to the invention is even distinctly greater than that of the adhesion barriers according to the prior art, the efficacy of which generally only amounts to around 60% (i.e. adhesions occur in approx. 40% of cases despite these adhesion barriers).

According to the invention, the non-woven fibre fabric may form the sole layer of the sheet material, i.e. no further layers are required for functionality as an adhesion barrier but may optionally be provided (see below). A non-woven fibre fabric (also known as fleece) is formed from a plurality of randomly arranged fibres, i.e. the fibres do not exhibit a preferential direction within the sheet material.

The non-woven fibre fabric is preferably produced by means of a rotary spinning method. Such a method and the resultant non-woven fabric of fibres of a crosslinked gelatin material are described in published patent application DE 10 2007 011 606 A1. Potential uses of the non-woven fibre fabric in medical applications which are mentioned therein are in particular wound coverings and carrier materials for living cells, but not use as an adhesion barrier in the field of surgery according to the present invention.

The gelatin material is favourably crosslinked subsequent to the actual production of the non-woven fibre fabric, in particular by the action of a crosslinking agent (for example formaldehyde) in the gas phase. Alternatively or additionally, the gelatin dissolved in the spinning solution for the rotary spinning method may already be crosslinked. All in all, this makes it possible to adjust the degree of crosslinking in a relatively targeted manner and so to adapt the absorption time of the adhesion barrier to the particular requirements of different application situations.

Due to its structure, the non-woven fibre fabric exhibits elevated flexibility, so simplifying handling by the surgeon and allowing the adhesion barrier to be adapted to the shape of the tissue in question. This flexibility means that the adhesion barrier can not only be used in conventional surgery, but can also be introduced laparoscopically into a patient as a medical implant.

Moreover, the non-woven fibre fabric exhibits relatively high porosity, as a result of which the individual fibres are relatively rapidly and effectively wettable. This wetting causes the adhesion barrier to adhere to the tissue with which it is brought into contact, such that it is in many cases unnecessary to fix it in place by suturing. Despite said adhesion of the wetted non-woven fibre fabric to tissue, the described function as an adhesion barrier is surprisingly achieved, i.e. unwanted adhesions between the two tissues separated by the adhesion barrier are counteracted. At higher levels of wetting, the non-woven fibre fabric changes over into a hydrated state in which a closed-pore, fibrous gel structure is obtained.

The gelatin material from which the fibres of the non-woven fabric are formed comprises a predominant proportion of gelatin, i.e. in particular at least 50 wt. %. In a preferred embodiment of the invention, the material comprises at least 90 wt. % gelatin or even substantially completely consists of gelatin (in each case relative to dry solids).

Alternatively, the gelatin material may, however, also comprise one or more further materials in order to have a purposeful influence on the properties of the adhesion barrier. Such materials may in particular be selected from chitosan, carrageen, alginate, pectin, starch and starch derivatives, cellulose and cellulose derivatives (for example CMC, HPMC, HEC and MC), modified gelatin (for example gelatin terephthalate, carbamoylate, succinate, dodecylsuccinate and acrylate) and gelatin copolymers (for example gelatin-polylactide conjugate).

The medical implant which is used according to the invention as an adhesion barrier preferably comprises a weight per unit area of 100 to 300 g/m², in particular of 180 to 220 g/m². The resultant weight per unit area may in particular be influenced by selection of the process parameters during production of the non-woven fibre fabric.

The thickness of the medical implant is favourably in the range from 1 to 6 mm, in particular in the range from 2.5 to 4.5 mm. With the same weight per unit area, the thickness of the non-woven fibre fabric may be reduced by mechanical compression (for example rollers), which is simultaneously accompanied by a reduction in porosity.

The fibres of the non-woven fabric preferably have an average diameter of 1 to 500 μm, in particular of 5 to 100 μm. Fibre thickness may likewise be influenced by various parameters of the manufacturing method (for example in a rotary spinning method). The structure of the non-woven fabric which is formed in part from very thin fibres is favourably stabilised in that the non-woven fabric comprises a plurality of regions in which two or more fibres merge into one another without a phase boundary. This is achieved during production by means of a rotary spinning method in that the individual fibres, when they come into contact with one another after leaving the spinneret, still have a relatively high water content and “fuse” together at the surface.

In a preferred embodiment of the invention, the non-woven fibre fabric has a density gradient along the thickness direction of the sheet material. In this case, the less dense (i.e. porous) side of the sheet material is brought into contact with a tissue in order to adhere to said tissue, while the adhesion between the denser (i.e. less porous) side of the sheet material and a tissue located on this side is lower. The adhesion barrier function is additionally promoted as a consequence.

In a further advantageous embodiment, the sheet material comprises two layers of non-woven fabrics of fibres of a crosslinked gelatin material, wherein the two layers have a different density. In this case too the lower density side is laid directly on a tissue, wherein the same effect is achieved as in the above-described variant with a density gradient.

According to a further preferred embodiment, the sheet material comprises a further layer which has a smooth, pore-free surface. Such a layer generally exhibits substantially lower adhesion to a tissue than a non-woven fibre fabric, since the lack of porosity means that wetting and hydration proceed more slowly. In this case, the layer with the non-woven fibre fabric is laid on a tissue and the smooth surface of the further layer may to a greater or lesser degree slide along another tissue. This further layer may here also act as a support for the non-woven fibre fabric and improve the stability and handling characteristics of the medical implant.

The further layer with the smooth, pore-free surface favourably comprises a film of a gelatin material. This makes it possible, despite the different functionalities, to produce the adhesion barrier from a uniform material. The production of suitable films from crosslinked gelatin is described, for example, in published patent application DE 10 2004 024 635 A1. The gelatin film may contain a plasticiser (for example glycerol) in order to achieve sufficient flexibility.

The adhesion barrier according to the present invention may in principle be used in any region of the body to prevent adhesion between tissues. Use in abdominal surgery is particularly preferred, in particular for preventing adhesions or coalescences between the abdominal wall and the interior organs (for example small intestine, large intestine, liver etc.). Further preferred fields of use are gynaecological surgery and urological surgery.

The present invention furthermore provides a method for preventing adhesion between a first tissue and a second tissue in a patient, comprising:
providing a medical implant in the form of a sheet material which comprises at least one layer of a non-woven fabric of fibres of a crosslinked gelatin material; and
applying the medical implant onto the first tissue during surgical treatment of the patient, such that the medical implant is arranged between the first tissue and the second tissue.

The first tissue preferably comprises a tissue of an internal organ in the abdominal region, in particular a tissue of the small intestine, the large intestine, the liver, the stomach, the spleen, the kidneys, the bladder, the uterus or the ovaries.

The second tissue in particular comprises a tissue of the abdominal wall, in particular of the peritoneum (parietal peritoneum).

Further advantages and preferred embodiments of the method according to the invention have already been described in connection with the use according to the invention of the medical implant as an adhesion barrier.

The purpose of the following Examples is to illustrate the present invention in greater detail.

### EXAMPLES

### Production of Non-Woven Fibre Fabrics

Non-woven fabrics of fibres of a crosslinked gelatin material were produced according to DE 10 2007 011 606 A1, to the full content of which reference is hereby made. A 20 wt. % aqueous solution of pigskin gelatin was here processed by means of a rotary spinning apparatus. The resultant gelatin fibres were collected on a laying apparatus. On laying, the fibres still have a sufficient water content for a non-woven fibre fabric to be able to form in which two or more fibres merge into one another without a phase boundary in a plurality of zones.

After drying, the non-woven fibre fabric was exposed for a period of approx. 8 hours at room temperature to the vapour pressure of a 10 wt. % formaldehyde solution in order to crosslink the gelatin. Conditioning then proceeded for approx. 3 days at a temperature of 50° C. and a relative humidity of 60% in order to complete the crosslinking reaction and remove excess formaldehyde. After this treatment, the residual content of formaldehyde was distinctly below the specified limit value of 200 mg/kg.

The non-woven fibre fabrics which were produced in this manner and used for the following tests, formed a sheet material with a weight per unit area in the range from 180 to 220 g/m² and a thickness in the range from 2.6 to 4.3 mm. The gelatin material of the fibres consists substantially completely of gelatin (relative to dry solids), i.e. the non-woven fabric exhibits excellent biocompatibility and may be produced both inexpensively and with a reproducible composition (elevated batch security).

### Use as an Adhesion Barrier in Animal Experimentation

The suitability as an adhesion barrier of non-woven fibre fabrics of a crosslinked gelatin material, which were produced according to the above Example, was tested in animal experiments on adult Lewis rats. The experimental animals were here divided into three groups each of 12 animals: the non-woven fibre fabric according to the invention was used in the first group, a prior art adhesion barrier (woven fabric of oxidised, regenerated cellulose) was used in the second group while the same intervention was carried out on the third group without an adhesion barrier (positive control).

The experimental animals (females, body weight 180 to 200 g) were in each case anaesthetised with a mixture of ketamine/xylazine and depilated over a large area of their ventral side. A laparotomy (opening the abdominal cavity) was carried out, an approx. 4 cm long skin incision initially being made at the level of the translucent linea alba, which was then severed with an incision 4 cm in length.

A 10×10 mm large lesion was then created on the abdominal peritoneal wall by abrasion with a scalpel until petechial bleeding occurred. After locating the caecum, a 10×10 mm large primary lesion was also induced here using a compress on the tissue surface of the end of the caecum, this latter lesion then being located opposite the lesioned area of the abdominal wall. It was ensured that slight haemorrhaging was already evident at this time.

For the purpose of testing the adhesion barriers, in the first group a non-woven fibre fabric of a crosslinked gelatin material and in the second group a woven fabric of oxidised, regenerated cellulose was in each case laid on the caecum prior to adaptation of the injured tissue. In the third group, the lesioned areas of the adjacent tissue were not separated from one another by an implant.

The caecum and peritoneum were then joined together in all the animals by means of three simple sutures. The knots were here located away from the edge of the injured areas.

After 28 days, a tensiometric measurement was carried out to investigate whether any unwanted adhesion was present between the two tissues. The caecum and peritoneum were here continuously pulled apart mechanically with increasing manual tensile force with the force required simultaneously being monitored and the tensile force required to separate the tissues was measured. In the presence of an adhesion, tensile forces in the range from 60 to 200 g were in each case necessary, while in those cases in which no adhesion was present the tissues could be separated from one another without application of a measurable force.

Evaluation of the three groups provided the following result:
First group: adhesions in 0 of 12 animals
Second group: adhesions in 3 of 12 animals
Third group: adhesions in 12 of 12 animals

At this time, the implants for both the first and the second groups had almost completely degraded. In no cases were inflammatory responses observed.

These results show that use according to the invention of a medical implant as an adhesion barrier can effectively prevent unwanted adhesion between two tissues resulting from surgical intervention, specifically with a distinctly higher success rate than in the case of an adhesion barrier according to the prior art.

1. A method for preventing adhesion between a first tissue and a second tissue in a body of a patient during surgical treatment, comprising:
providing a medical implant in the form of a sheet material which comprises at least one layer of a non-woven fabric of fibres of a crosslinked gelatin material; and
applying the medical implant onto the first tissue in the body of the patient such that the medical implant is arranged between the first tissue and the second tissue.

2. The method according to claim 1, wherein the non-woven fibre fabric is produced by a rotary spinning method.

3. The method according to claim 1, wherein the gelatin material comprises at least 50 wt. % of crosslinked gelatin.

4. The method according to claim 1, wherein the medical implant has a weight per unit area of 100 to 300 g/m².

5. The method according to claim 1, wherein the medical implant has a thickness of 1 to 6 mm.

6. The method according to claim 1, wherein the fibres of the non-woven fabric have an average diameter of 1 to 500 μm.

7. The method according to claim 1, wherein the non-woven fibre fabric has a density gradient along a thickness direction of the sheet material.

8. The method according to claim 1, wherein the sheet material comprises two layers of non-woven fabrics of fibres of a crosslinked gelatin material, and wherein the two layers have different densities.

9. The method according to claim 1, wherein the sheet material comprises a further layer which has a smooth, pore-free surface.

10. The method according to claim 9, wherein the further layer comprises a film of a gelatin material.

11. The method according to claim 1, wherein the surgical treatment comprises abdominal surgery, gynaecological surgery or urological surgery.

12. The method according to claim 3, wherein the gelatin material comprises at least 90 wt. % of crosslinked gelatin.

13. The method according to claim 4, wherein the medical implant has a weight per unit area of 180 to 220 g/m².

14. The method according to claim 5, wherein the medical implant has a thickness of 2.5 to 4.5 mm.

15. The method according to claim 6, wherein the fibres of the non-woven fabric have an average diameter of 5 to 100 μm.
